SEQUENCE LISTING (63 sequences)

SEQ ID NO: 1 - protein, 19 aa, Artificial Sequence
  Synthetic peptide
LSAFDERRNKYLEEHPSAG

SEQ ID NO: 2 - protein, 10 aa, Artificial Sequence
  Synthetic peptide
LSAFDERRNK

SEQ ID NO: 3 - protein, 10 aa, Artificial Sequence
  Synthetic peptide
AFDERRNKYL

SEQ ID NO: 4 - protein, 10 aa, Artificial Sequence
  Synthetic peptide
DERRNKYLEE

SEQ ID NO: 5 - protein, 10 aa, Artificial Sequence
  Synthetic peptide
RRNKYLEEHP

SEQ ID NO: 6 - protein, 10 aa, Artificial Sequence
  Synthetic peptide
NKYLEEHPSA

SEQ ID NO: 7 - protein, 10 aa, Artificial Sequence
  Synthetic peptide
KYLEEHPSAG

SEQ ID NO: 8 - protein, 32 aa, Artificial Sequence
  Synthetic peptide
ALNGNGDPNNMDKAVKLYRKLKREITFHGAKE

SEQ ID NO: 9 - protein, 29 aa, Artificial Sequence
  Synthetic peptide
GNGDPNNMDKAVKLYRKLKREITFHGAKE

SEQ ID NO: 10 - protein, 15 aa, Artificial Sequence
  Synthetic peptide
ALNGNGDPNNMDKAV

SEQ ID NO: 11 - protein, 15 aa, Artificial Sequence
  Synthetic peptide
NGNGDPNNMDKAVKL

SEQ ID NO: 12 - protein, 15 aa, Artificial Sequence
  Synthetic peptide
NGDPNNMDKAVKLYR

SEQ ID NO: 13 - protein, 15 aa, Artificial Sequence
  Synthetic peptide
DPNNMDKAVKLYRKL

SEQ ID NO: 14 - protein, 15 aa, Artificial Sequence
  Synthetic peptide
NNMDKAVKLYRKLKR

SEQ ID NO: 15 - protein, 15 aa, Artificial Sequence
  Synthetic peptide
MDKAVKLYRKLKREI

SEQ ID NO: 16 - protein, 15 aa, Artificial Sequence
  Synthetic peptide
KAVKLYRKLKREITF

SEQ ID NO: 17 - protein, 15 aa, Artificial Sequence
  Synthetic peptide
VKLYRKLKREITFHG

SEQ ID NO: 18 - protein, 15 aa, Artificial Sequence
  Synthetic peptide
LYRKLKREITFHGAK

SEQ ID NO: 19 - protein, 15 aa, Artificial Sequence
  Synthetic peptide
YRKLKREITFHGAKE

SEQ ID NO: 20 - protein, 9 aa, Artificial Sequence
  Synthetic peptide
GILGFVFTL

SEQ ID NO: 21 - protein, 9 aa, Artificial Sequence
  Synthetic peptide
IMDQVPFSV

SEQ ID NO: 22 - protein, 9 aa, Artificial Sequence
  Synthetic peptide
ITDQVPFSV

SEQ ID NO: 23 - DNA, 21 bp, Artificial Sequence
  Synthetic oligonucleotide
ttaatacgactcactataggg

SEQ ID NO: 24 - DNA, 18 bp, Artificial Sequence
  Synthetic oligonucleotide
tagaaggcacagtcgagg

SEQ ID NO: 25 - DNA, 47 bp, Artificial Sequence
  Synthetic oligonucleotide
ttacagggtgaacacgaagcccaggatgccgaagtagctgccctcgt

SEQ ID NO: 26 - DNA, 20 bp, Artificial Sequence
  Synthetic oligonucleotide
ttactgtccagcgctagccc

SEQ ID NO: 27 - DNA, 47 bp, Artificial Sequence
  Synthetic oligonucleotide
ttacagggtgaacacgaagcccaggatgccctgtccagcgctagccc

SEQ ID NO: 28 - DNA, 21 bp, Artificial Sequence
  Synthetic oligonucleotide
ttaccggaaggggtcgatgcc

SEQ ID NO: 29 - DNA, 20 bp, Artificial Sequence
  Synthetic oligonucleotide
ttatctccaaaaattccggt

SEQ ID NO: 30 - DNA, 47 bp, Artificial Sequence
  Synthetic oligonucleotide
ttacagggtgaacacgaagcccaggatgcctctccaaaaattccggt

SEQ ID NO: 31 - DNA, 20 bp, Artificial Sequence
  Synthetic oligonucleotide
ttacagctcccgcatccact

SEQ ID NO: 32 - DNA, 47 bp, Artificial Sequence
  Synthetic oligonucleotide
ttacagggtgaacacgaagcccaggatgcctccggcgctggggtgtt

SEQ ID NO: 33 - DNA, 20 bp, Artificial Sequence
  Synthetic oligonucleotide
ttaccgtctttcgtcgaagg

SEQ ID NO: 34 - DNA, 47 bp, Artificial Sequence
  Synthetic oligonucleotide
ttacagggtgaacacgaagcccaggatgccccgtctttcgtcgaagg

SEQ ID NO: 35 - DNA, 20 bp, Artificial Sequence
  Synthetic oligonucleotide
ttagatgtagaaccggccga

SEQ ID NO: 36 - DNA, 47 bp, Artificial Sequence
  Synthetic oligonucleotide
ttacagggtgaacacgaagcccaggatgccgatgtagaaccggccga

SEQ ID NO: 37 - DNA, 20 bp, Artificial Sequence
  Synthetic oligonucleotide
ttacttgaaccgctgcatct

SEQ ID NO: 38 - DNA, 47 bp, Artificial Sequence
  Synthetic oligonucleotide
ttacacgctgaagggcacctggtccatgatcttgaaccgctgcatct

SEQ ID NO: 39 - DNA, 20 bp, Artificial Sequence
  Synthetic oligonucleotide
ttagctgctgccggccatct

SEQ ID NO: 40 - DNA, 47 bp, Artificial Sequence
  Synthetic oligonucleotide
ttacacgctgaagggcacctggtccatgatgctgctgccggccatct

SEQ ID NO: 41 - DNA, 20 bp, Artificial Sequence
  Synthetic oligonucleotide
ttaacacaccaggccgaagg

SEQ ID NO: 42 - DNA, 47 bp, Artificial Sequence
  Synthetic oligonucleotide
ttacacgctgaagggcacctggtccatgatacacaccaggccgaagg

SEQ ID NO: 43 - DNA, 20 bp, Artificial Sequence
  Synthetic oligonucleotide
ttaggccttgtccatgttgt

SEQ ID NO: 44 - DNA, 47 bp, Artificial Sequence
  Synthetic oligonucleotide
ttacacgctgaagggcacctggtccatgatggccttgtccatgttgt

SEQ ID NO: 45 - DNA, 20 bp, Artificial Sequence
  Synthetic oligonucleotide
ttagtagatcaggcccatgc

SEQ ID NO: 46 - DNA, 47 bp, Artificial Sequence
  Synthetic oligonucleotide
ttacacgctgaagggcacctggtccatgatgtagatcaggcccatgc

SEQ ID NO: 47 - DNA, 20 bp, Artificial Sequence
  Synthetic oligonucleotide
ttactctttggcgccgtgga

SEQ ID NO: 48 - DNA, 47 bp, Artificial Sequence
  Synthetic oligonucleotide
ttacacgctgaagggcacctggtccatgatctctttggcgccgtgga

SEQ ID NO: 49 - DNA, 20 bp, Artificial Sequence
  Synthetic oligonucleotide
ttacagccattccatcagca

SEQ ID NO: 50 - DNA, 47 bp, Artificial Sequence
  Synthetic oligonucleotide
ttacacgctgaagggcacctggtccatgatcagccattccatcagca

SEQ ID NO: 51 - DNA, 20 bp, Artificial Sequence
  Synthetic oligonucleotide
ttacagggtgaacacgaagc

SEQ ID NO: 52 - DNA, 47 bp, Artificial Sequence
  Synthetic oligonucleotide
ttacacgctgaagggcacctggtccatgatcagggtgaacacgaagc

SEQ ID NO: 53 - DNA, 20 bp, Artificial Sequence
  Synthetic oligonucleotide
ttacttggtcagggggctca

SEQ ID NO: 54 - DNA, 47 bp, Artificial Sequence
  Synthetic oligonucleotide
ttacacgctgaagggcacctggtccatgatcttggtcagggggctca

SEQ ID NO: 55 - DNA, 19 bp, Artificial Sequence
  T7 promoter sequence
taatacgactcactatagg

SEQ ID NO: 56 - DNA, 19 bp, Artificial Sequence
  T3 promoter sequence
aattaaccctcactaaagg

SEQ ID NO: 57 - DNA, 23 bp, Artificial Sequence
  T3 promoter sequence
aattaaccctcactaaagggaga

SEQ ID NO: 58 - DNA, 19 bp, Artificial Sequence
  SP6 promoter sequence
atttaggtgacactataga

SEQ ID NO: 59 - DNA, 23 bp, Artificial Sequence
  SP6 promoter sequence
atttaggtgacactatagaagng

SEQ ID NO: 60 - protein, 20 aa, Homo sapiens
MDLVLKRCLLHLAVIGALLA

SEQ ID NO: 61 - protein, 21 aa, Homo sapiens
QVPLIVGILLVLMAVVLASLI

SEQ ID NO: 62 - protein, 22 aa, Homo sapiens
IYIWAPLAGTCGVLLLSLVITL

SEQ ID NO: 63 - protein, 10 aa, Artificial Sequence
  Synthetic peptide
YRKLKREITF

(19) United States
(12) Patent Application Publication
(10) Pub. No.: US 2013/0085260 A1
(43) Pub. Date: Apr. 4, 2013

(54) METHOD FOR THE IDENTIFICATION OF T CELL EPITOPES

(71) Applicants: Réjean Lapointe, Laval (CA); Jean-Daniel Doucet, Quebec (CA)

(72) Inventors: Réjean Lapointe, Laval (CA); Jean-Daniel Doucet, Quebec (CA)

(73) Assignee: CENTRE HOSPITALIER DE L'UNIVERSITE DE MONTREAL, Montreal, QC (CA)

(21) Appl. No.: 13/642,685

(22) PCT Filed: Apr. 21, 2011
(86) PCT No.: PCT/CA2011/050227

§ 371 Date: Nov. 21, 2012

Related U.S. Application Data
(60) Provisional application No. 61/326,784, filed on Apr. 22, 2010.

(51) Int. Cl. C12Q 1/02

(52) U.S. Cl. CPC C12Q 1/02
USPC 530/324; 435/29; 530/328; 530/327; 530/326; 530/325

(57) ABSTRACT

A novel method to identify relevant T-cell epitopes recognized by CD8⁺ or CD4⁻ T lymphocytes is described. The method is based on the use of mRNA fragments synthesized from cDNA encoding portions of a polypeptide of interest. mRNA fragments are introduced into antigen-presenting cells to deduce an epitope's localization in a polypeptide of interest, such as a protein antigen.

### CROSS REFERENCE TO RELATED APPLICATIONS

This application claims the benefit of U.S. provisional application Ser. No. 61/326,784, filed on Apr. 22, 2010, which is incorporated herein by reference in its entirety.

### SEQUENCE LISTING

This application contains a Sequence Listing in computer readable form entitled 12810_{—}404—sequence listing_ST25, created Apr. 21, 2011 and having a size of 13.5 kb, which is incorporated herein by reference.

### TECHNICAL FIELD

The present invention generally relates to the field of immunology and vaccines, and more particularly to the identification of T cell epitopes.

### BACKGROUND ART

Vaccine development is one of the priorities defined by the World Health Organization. There is a clear need in both viral¹ and tumor² immunology to find a wide array of antigens that can be targeted by both immune CD8⁺ cytotoxic and CD4⁺ helper T cells recognizing epitopes presented by major histocompatibility complex (MHC) classes I and II. Several approaches have been developed to identify these T cell peptide epitopes¹. So far, the synthesis of vast peptide libraries has allowed the identification of many T cell epitopes presented by MHC class I and II in different diseases. Unfortunately, this technique involves the synthesis and screening of an large number of peptides, which is time-consuming, expensive and tedious. Bioinformatics epitope³ and proteasome cleavage site predictions might reduce the number of peptides tested but they are still far from being accurate. Recently-described ultraviolet light-dependent MHC-peptide exchange technology^{4,5} could also accelerate epitope identification. Still, synthetic peptides do not take into account for example epitopes coded by alternative reading frames⁶ or post-translationally-modified epitopes⁷ and epitopes generated by protein splicing⁸. Synthetic peptides may also identify irrelevant cryptic epitopes that are immunogenic in peptide form but are not processed in vivo by antigen-presenting cells (APCs)⁹.

Another epitope identification strategy consists of analyzing, by mass spectrometry, peptides bound to MHC molecules¹⁰. While this strategy is high throughput, the peptides identified may not necessarily reflect genuine epitopes recognized by specific T lymphocytes¹¹. Another technique is involves digestion of a plasmid to find T cell epitope-containing regions¹². The plasmid templates are cleaved at different sites with restriction enzymes. The technique employs restriction sites that are randomly distributed in the genome. A long process of site-specific mutagenesis and subsequent subcloning may often be required to insert restriction sites where needed. Moreover, this technique exploits the K562 cell line stably transfected with a defined HLA molecule as APCs, which may not reflect the full haplotype of an individual.

There is thus a need for the development of novel strategies to identify T cell epitopes.

The present description refers to a number of documents, the content of which is herein incorporated by reference in their entirety.

### SUMMARY OF THE INVENTION

In a first aspect, the present invention provides a method for determining whether a region of a polypeptide of interest comprises one or more T cell epitopes, said method comprising: (a) providing an mRNA comprising a first domain encoding said region, wherein said mRNA is obtained by in vitro transcription of a DNA encoding said region, and wherein said DNA is obtained by nucleic acid amplification using one or more oligonucleotides hybridizing to a nucleic acid encoding said polypeptide of interest or to the complement thereof; (b) introducing said mRNA into an antigen-presenting cell (APC) population; and (c) determining the ability of said APC population to activate a first T cell population; wherein activation of said first T cell population by said APC population is indicative that said region comprises one or more T cell epitopes.

In another aspect, the present invention provides a method for determining whether a region of a polypeptide of interest comprises one or more T cell epitopes, said method comprising: (a) providing a first mRNA comprising a first domain encoding said polypeptide of interest or a fragment thereof comprising said region; (b) providing a second mRNA comprising the first domain of said first mRNA but in which the portion encoding said region is lacking, wherein said first and second mRNA are obtained by in vitro transcription of DNAs encoding said polypeptide of interest or fragment thereof, and wherein said DNAs are obtained by nucleic acid amplification using oligonucleotides hybridizing to different portions of a nucleic acid encoding said polypeptide of interest or a complement thereof; (c) introducing said first and second mRNAs into first and second antigen-presenting cell (APC) populations, respectively; and (d) determining the ability of said first and second APC populations to activate a first T cell population; wherein a higher activation of said first T cell population by said first APC population relative to said second APC population is indicative that said region comprises one or more T cell epitopes.

In an embodiment, the above-mentioned nucleic acid encoding said polypeptide of interest is comprised within a plasmid.

In an embodiment, the above-mentioned nucleic acid amplification is polymerase chain reaction (PCR).

In an embodiment, the above-mentioned region comprises from about 10 to about 100 amino acids, in a further embodiment from about 15 to about 50 amino acids.

In an embodiment, the above-mentioned second mRNA encodes a C-terminal deletion mutant of the polypeptide of interest or fragment thereof of (a).

In an embodiment, the above-mentioned mRNA further comprise a second domain encoding a detectable moiety, and wherein said method further comprises determining the presence of said detectable moiety. In a further embodiment, the above-mentioned detectable moiety is a known T cell epitope, and wherein said method further comprises determining the ability of said APC populations to activate a second T cell population recognizing said known T cell epitope.

In an embodiment, the above-mentioned first and second mRNAs further comprise a second domain encoding a known T cell epitope, and wherein said method further comprises determining the ability of first and second APC populations to activate a second T cell population recognizing said known T cell epitope.

In an embodiment, the above-mentioned second domain is located 3′ relative to said first domain.

In an embodiment, the above-mentioned mRNA further comprising a poly(A) tail.

In an embodiment, the above-mentioned APC is a B-cell.

In an embodiment, the above-mentioned first T cell population is a T cell clone.

In a further embodiment, the above-mentioned T cell clone is derived from peripheral blood T cells stimulated with said polypeptide of interest or a fragment thereof in the presence of APCs.

In an embodiment, the above-mentioned APC population and said first T cell population are autologous.

In an embodiment, the above-mentioned introducing is through electroporation.

In another aspect, the present invention provides a method for identifying one or more T cell epitopes in a polypeptide of interest, said method comprising: (a) performing the above-mentioned method to identify a region of said polypeptide comprising said one or more T cell epitopes; (b) contacting a T cell population with an antigen-presenting cell (APC) population loaded or pulsed with a peptide comprising a sequence of amino acids from said region, wherein said peptide comprises at least 7 amino acids; (c) determining the ability of said APC population to activate said T cell population; and (d) identifying the T cell epitope in accordance with said determination.

In an embodiment, a plurality of different peptides comprising amino acids located within said region loaded on a plurality of APC populations are used, wherein each of said APC populations is loaded with a different peptide. In a further embodiment, the above-mentioned plurality of peptides are overlapping peptides encompassing the entire region.

In another aspect, the present invention provides a peptide of 50 amino acids or less comprising the amino acid sequence of SEQ ID NOs: 3, 11 or 63.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the validation of the mRNA PCR-based epitope chasing (mPEC) approach with a defined HLA-A*0201 epitope from M1 influenza protein (M1⁵⁸⁻⁶⁶, GILGFVFTL, SEQ ID NO: 20). Epstein-Barr virus (EBV)-B cells were electroporated with empty or M1-coding DNA plasmids, or with mRNA prepared from PCR-amplified M1 or mock (Ctl) plasmid cDNA, with the gp100²⁶⁹⁻²¹⁸-coding sequence epitope inserted in the 3′end primer (g209). EBV-B cells were also directly pulsed with peptides corresponding to M1⁵⁸⁻⁶⁶ or gp100²⁰⁹⁻²¹⁸epitopes. Presentation of relevant epitopes was assessed by co-culture with either M1⁵⁸⁻⁶⁶ (M1-CD8; light grey, upper bars) or gp100²⁰⁹⁻²¹⁸ (black, lower bars)-specific T cells. IFN-γ production was quantified by enzyme-linked immunosorbent assay (ELISA) (range <16 to >5,000 pg/ml, error bars, SD), representative of 3 independent experiments. The M1 fragment sizes indicated in the legend are approximated. The M1⁵⁸⁻⁶⁶ epitope to which the M1-CD8 T cell clone is specific is indicated by an oval;

FIG. 2 shows the identification of unknown major histocompatibility complex (MHC) class I and class II epitopes from influenza antigens by the mRNA polymerase chain reaction-based epitope chase method. EBV-B cells were electroporated with mRNA prepared from PCR-amplified NP (panel A), M1 (panel C), or mock (Ctl) cDNA, with respectively the M1⁵⁸⁻⁶⁶ (panel A, light grey bars) or gp100^{209-218/2M}-(panel C, black bars) coding sequence epitope added at the 3′end of mRNAs. EBV-B cells were also pulsed with NP-CD8 (panel B) or M1-CD4 (panel D) peptides (see Table 2 for the list of peptides). Presentation of relevant epitopes was assessed by co-culture with either NP-CD8 (panels A, B, black bars) and M1⁵⁸⁻⁶⁶ (panel A, light grey, lower bars) or M1-CD4 (panels C, D, black bars) and gp100^{209-218/2M}-(panel C, black, upper bars) specific T-cell clones. Interferon-γ (IFN-γ) production was quantified by enzyme-linked immunosorbent assay [range: <16 to >10,000 pg/ml (panels A, C); <16 to 60,000 pg/mL (panels B, D), error bars, SD], representative of 3 independent experiments. The NP and M1 fragment sizes indicated in the legend are approximated. The NP-CD8 and M1-CD4 epitopes are indicated by an oval;

FIG. 3 shows mRNA preparation from PCR-amplified cDNA. (A) Schematic representation of M1 or NP synthetic mRNA fragments prepared from PCR-amplified cDNA and co-culture of electroporated autologous EBV-B cells with specific T cells. (B) M1 PCR-amplified cDNA fragments with or without 3′end gp100^{209-218/2M} control peptide (g209) were migrated on 1.5% agarose gel for 1 h. (C) Migration of some M1 RNA fragments synthesized from M1 cDNA fragment templates, with or without subsequent poly-adenylation, was performed for 15 min on 1.5% agarose gel in non-denaturing, non-RNase-free conditions. The same controls were used for NP fragments and for all other fragments;

FIG. 4 shows the identification of MHC class I and class II epitopes from influenza antigens by the mPEC method in the absence of 3′end control epitopes. EBV-B cells were electroporated with mRNA prepared from PCR-amplified (A) NP or (C) M1 cDNA. EBV-B cells were also directly pulsed with M1⁵⁸⁻⁶⁶ or gp100^{209-218/2M} peptides (second from bottom and bottom-most results of panel A, respectively). Presentation of relevant epitopes was evaluated by co-culture with either (A) M1-CD8, (B) NP-CD8 or (C) M1-CD4 specific T cells. IFN-γ production was assessed by ELISA (range <16 to >5,000 pg·ml⁻¹), representative of 3 independent experiments. The NP and M1 fragment sizes indicated in the legend are approximated. Each T cell clone epitope is delineated by an oval;

FIG. 5 shows that EBV-B and CD40-B cells are competent in presenting MHC class I epitopes after RNA or DNA electroporation. EBV-B or CD40-activated B cells were electroporated with M1 coding DNA plasmids or mRNA prepared from PCR-amplified M1 cDNA. Presentation of relevant epitopes was assessed by co-culture with M1⁵⁸⁻⁶⁶-specific T cells. IFN-γ production was quantified by ELISA.

### DISCLOSURE OF INVENTION

The present inventors have developed a novel mRNA epitope identification method to rapidly and precisely identify relevant T-cell epitopes recognized by CD8⁺ and/or CD4⁺ T lymphocytes. This method is based on the use of mRNA synthesized from a DNA encoding a polypeptide of interest or a portion thereof. The mRNA is introduced into antigen-presenting cells whereby it may be determined whether the encoded polypeptide or portion thereof is capable of T-cell activation, and in turn it may be determined whether the polypeptide or portion thereof comprises such an epitope. Further, such analysis of different portions of the polypeptide allows for the epitope's localization in the polypeptide (e.g., a protein antigen) or portion thereof.

Accordingly, in a first aspect, the present invention provides a method for determining whether a region of a polypeptide of interest comprises one or more T cell epitopes, said method comprising:
providing an mRNA comprising a first domain encoding said region, wherein said mRNA is obtained by in vitro transcription of a DNA encoding said region, and wherein said DNA is obtained by nucleic acid amplification using one or more oligonucleotides hybridizing to a nucleid acid encoding said polypeptide of interest or to the complement thereof;
introducing said mRNA into an antigen-presenting cell (APC) population; and
determining the ability of said APC population to activate a first T cell population;
   wherein activation of said first T cell population by said APC population is indicative that said region comprises one or more T cell epitopes.

In another aspect, the present invention provides a method for determining whether a region of a polypeptide of interest comprises one or more T cell epitopes, said method comprising:

(a) providing a first mRNA comprising a first domain encoding said polypeptide of interest or a fragment thereof comprising said region;

(b) providing a second mRNA comprising the first domain of said first mRNA but in which the portion encoding said region is lacking, wherein said first and second mRNA are obtained by in vitro transcription of DNAs encoding said polypeptide of interest or fragment thereof, and wherein said DNAs are obtained by nucleic acid amplification using oligonucleotides hybridizing to different portions of a nucleic encoding said polypeptide of interest or a complement thereof;

(c) introducing said first and second mRNAs into first and second antigen-presenting cell (APC) populations, respectively; and

(d) determining the ability of said first and second APC populations to activate a first T cell population;

wherein a higher activation of said first T cell population by said first APC population relative to said second APC population is indicative that said region comprises one or more T cell epitopes.

The term “polypeptide of interest” as used herein refers to any polypeptide for which the identification of T-cell epitopes and/or of regions comprising same is desired. The polypeptide may be of any origin (e.g., viral, bacterial, parasital, fungal, tumoral), and may comprise the entire coding sequence of a naturally occurring protein, or a fragment thereof. The term “T-cell epitope” refers to peptides that can bind to MHC class I and II molecules and that are capable of inducing activation of CD8⁺ (CD8⁺ T cell epitopes) and/or CD4⁺ (CD4⁺ T cell epitopes) T cells. CD8⁺ T cell epitopes, bound to MHC class I molecules, are typically peptides between about 8 and about 11 amino acids in length, whereas CD4⁺ T cell epitopes, bound to MHC class II molecules, are of more variable length, but are typically from about 13 to about 25 amino acids.

The term “region” as used herein (in reference to a polypeptide of interest) includes the entire coding sequence of a polypeptide of interest (e.g., a naturally-occurring protein), or any portion thereof. In an embodiment, the above-mentioned region comprises from about 10 to about 100 amino acids, in a further embodiment from about 15 to about 50 amino acids (e.g., 15, 20, 25, 30, 35, 40, 45 or 50 amino acids) of the polypeptide of interest. Thus, a polypeptide of interest may be divided into small regions (and the above-mentioned method repeated for each individual region), which permits a more precise mapping of the localization of the epitope(s).

The above-mentioned mRNA is obtained by in vitro transcription of a cDNA. Methods for in vitro synthesis of mRNA using RNA polymerases (the most common RNA polymerases used are SP6, T7 and T3 polymerases) are well known in the art and kits for doing so are commercially available from several providers, including the MEGAscript® High Yield Transcription Kit and mMESSAGE mMACHINE® High Yield RNA Transcription Kit from Ambion, Inc., the HiScribe™ T7 In Vitro Transcription Kit from New England BioLabs Inc. and the TranscriptAid™ T7 High Yield Transcription Kit from Thermo Scientific.

The DNA used for in vitro transcription is prepared by nucleic acid amplification (e.g., PCR) using a nucleic acid (e.g., DNA) encoding the polypeptide of interest, or a fragment thereof, as a template, and one or more oligonucleotides (primers) specifically hybridizing to the nucleic acid encoding the polypeptide of interest or to the complement thereof. In an embodiment, the DNA template is comprised/cloned in a plasmid. The DNA also comprises at its 5′ end a promoter region operably linked to the first domain that allows binding to RNA polymerase (e.g., a T3, T7 or SP6 promoter region) and subsequent transcription of the DNA to generate the above-mentioned mRNA. In an embodiment, the promoter region is incorporated into the DNA using a forward primer comprising such a promoter region for DNA amplification. In an embodiment, the DNA template is comprised/cloned in a plasmid that contains a promoter region sequence of a RNA polymerase (e.g., a T3, T7 or SP6 promoter region), and the forward primer used for amplification comprises a sequence specifically hybridizing to the promoter region sequence or to the complement thereof. In an embodiment, the T7 promoter region sequence comprises the following sequence TAATACGACTCACTATAGG (SEQ ID NO: 55), in a further embodiment TTAATACGACTCACTATAGGG (SEQ ID NO: 23). In an embodiment, the T3 promoter region sequence comprises the following sequence AATTAACCCTCACTAAAGG (SEQ ID NO: 56), in a further embodiment AATTAACCCTCACTAAAGGGAGA (SEQ ID NO: 57). In an embodiment, the SP6 promoter region sequence comprises the following sequence ATTTAGGTGACACTATAGA (SEQ ID NO: 58), in a further embodiment ATTTAGGTGACACTATAGAAGNG (SEQ ID NO: 59). The DNA also comprises at its 3′ end a stop codon.

In some embodiments, the above-mentioned oligonucleotides comprise from about 10 to about 100 nucleotides, in further embodiments from about 15 to about 100, from about 15 to about 50, from about 15 to about 40, from about 15 to about 30 nucleotides.

The term “specifically hybridizing” refers to the association between two single-stranded nucleotide molecules of sufficiently complementary sequence to permit such hybridization under predetermined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art. For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. A moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

In an embodiment, the above-mentioned mRNA further comprises a second domain encoding a detectable moiety, and the above-mentioned method further comprises determining the presence of the detectable moiety. The second domain may be localized 5′ or 3′ relative to the first domain. In an embodiment, the second domain is 3′ relative to the first domain. The detectable moiety is useful as a positive control for mRNA quality and/or transfection efficiency, i.e. the presence of the detectable moiety being indicative that the mRNA is not altered or degraded, and that the APCs were transfected. The detectable moiety may be any polypeptide or peptide whose expression or presence may be detected, such as an enzyme, a fluorescent polypeptide, a known peptide/epitope specifically recognized by a specific antibody or ligand (e.g., peptide tags commonly used in affinity purification such as His, CBP, CYD, Strep II, FLAG and HPC peptide tags) or a known T cell epitope that may be detected using a T cell recognizing the epitope (e.g., a T cell clone, hybridoma or line). In an embodiment, the above-mentioned detectable moiety is a known T cell epitope, and said method further comprises determining the ability of the APC population to activate a second T cell population (e.g., a T cell clone, hybridoma or line) recognizing said known T cell epitope. Any known epitope for which an epitope-specific T cell population (e.g., a T cell line, clone or hybridoma) is available, or may be easily generated, may be used in the above-mentioned method. An example of such known epitope is the native (ITDQVPFSV, SEQ ID NO: 22) and optimized (IMDQVPFSV, SEQ ID NO: 21) versions of the gp100 HLA-A*0201-restricted epitope (209-218), which is recognized by the known gp100-specific CD8⁺ (g209) T-cell clone. Another example is the influenza A virus matrix protein peptide 58-66 (M1⁵⁸⁻⁶⁶), which is recognized by M1⁵⁸⁻⁶⁶-specific T cells.

In an embodiment, the above-mentioned mRNA further comprises a poly(A) tail. Methods for polyadenylating mRNA are well known in the art and kits for doing so are commercially available from several providers, including the Poly(A) Tailing Kit from Ambion, Inc., and the Poly(A) Polymerase Tailing Kit from EPICENTRE Biotechnologies.

In an embodiment, the above-mentioned mRNA further comprises a third domain encoding an MHC class II compartment mobilization sequence¹⁹⁻²¹, which may increase the processing and presentation of CD4⁺ T cell epitopes by MHC class II molecules for certain polypeptides/antigens. Such MHC class II compartment mobilization sequences include, for example, sequences encoding signal peptides and/or transmembrane domains²¹. In an embodiment, the sequence encoding a signal peptide is that of gp100 (MDLVLKRCLLHLAVIGALLA, SEQ ID NO: 60). In another embodiment, the sequence encoding a transmembrane domain is that of gp100 (QVPLIVGILLVLMAVVLASLI, SEQ ID NO: 61) or CD8 (IYIWAPLAGTCGVLLLSLVITL, SEQ ID NO: 62).

In an embodiment, the above-mentioned second mRNA is a truncation or deletion mutant of the first domain, i.e., in which the sequence encoding the region studied has been deleted. Such deletion may be a C-terminal deletion (i.e., a truncation), an N-terminal deletion (i.e., a truncation) or an internal deletion. In an embodiment, the deletion is a C-terminal deletion. In an embodiment, the deletion is a deletion of about 10 to about 100 amino acids, in a further embodiment from about 15 to about 50 amino acids (e.g., 15, 20, 25, 30, 35, 40, 45 or 50 amino acids).

In another embodiment, the above-mentioned first mRNA is an addition or insertion mutant of the second domain, i.e. in which the sequence encoding the region studied has been added. Such addition may be a C-terminal addition, an N-terminal addition or an internal addition. In an embodiment, the deletion is a C-terminal addition. In another embodiment, the addition is an addition of about 10 to about 100 amino acids, in a further embodiment from about 15 to about 50 amino acids (e.g., 15, 20, 25, 30, 35, 40, 45 or 50 amino acids).

The term “antigen-presenting cell (APC)” as used herein refers to any cell capable of processing and presenting an antigen via an MHC molecule (MHC class I and/or MHC class II molecules). In an embodiment, the APC is capable of processing and presenting an antigen via MHC class I and MHC class II molecules. In a further embodiment, the APC is a dendritic cell, a macrophage or a B-cell. In yet a further embodiment, the APC is a B-cell. In another embodiment, the B-cell is immortalized and/or activated.

In an embodiment, the above-mentioned first T cell population is a T cell clone, in a further embodiment a T cell clone derived from peripheral blood T cells stimulated with said polypeptide of interest, or a fragment thereof, in the presence of APCs (e.g., dendritic cells, B-cells)⁸. Methods to generate a T cell clone are well known in the art and include, for example, limiting dilution (LD), and cell sorting (e.g., fluorescence-activated cell sorting or FACS, magnetic affinity cell sorting or MACS).

In an embodiment, the above-mentioned APC population and first T cell population are autologous (i.e. are derived from cells from the same individual). In another embodiment, the above-mentioned APC population, first T cell population and second T cell population are autologous.

In an embodiment, the above-mentioned APC and/or T cell populations are of human origin.

The above-mentioned mRNA may be introduced/incorporated into the APCs using any cell transfection, transformation or transduction method, including, for example, microinjection, electroporation, and lipid-mediated transfection methods. Kits and reagents for incorporating mRNA into cells are commercially available, from several providers, including the TransMessenger™ Transfection Reagent from Qiagen and the TransIT®-mRNA Transfection Kit from Mirus. In an embodiment, the above-mentioned mRNA is incorporated through electroporation.

The ability of an APC population to activate a T cell population may be determined using any methods/assays for measuring T cell activation/stimulation including, for example, (i) the secretion of cytokines (e.g., IL-2, IFN-γ) or other molecules associated with T cell activation (e.g., chemokines) by ELISA, ELISPOT or flow cytometry, (ii) T cell proliferation by ³H-thymidine incoporation or CFSE dilution, (iii) expression of activation markers at the T cell surface, (iv) expression of genes associated with T cell activation (e.g., using DNA or protein microarray), (v) cytotoxicity, and (vi) assessment of signalling pathways/mediators in the T cell (e.g., phosphorylation status, calcium flux/levels). In an embodiment, the ability of the APC population to activate the T cell population is determined by measuring the secretion of IFN-γ by the T cells. In a further embodiment, the secretion of IFN-γ is measured by ELISA. A “higher” activation of a first T cell population relative to a second T cell population refers to an activation that is at least 10%, 20%, 30%, 40%, 50%, 100% or 200% higher in the first T cell population relative to the second T cell population, as determined using any method for measuring T cell activation, such as those mentioned above.

While the above-mentioned method may potentially permit to identify one or more epitopes in the polypeptide of interest (especially if the polypeptide of interest is divided into several small regions), further delineation of the epitope comprised within the region identified by the above-mentioned method may involve a further mapping step using one or more peptides comprising amino acids from this region.

Accordingly, in another aspect, the present invention provides a method for identifying one or more T cell epitopes in a polypeptide of interest, said method comprising:

performing the above-mentioned method to identify a region of said polypeptide comprising said one or more T cell epitopes;

contacting a T cell population with an antigen-presenting cell (APC) population loaded or pulsed with a peptide comprising a sequence of amino acids from said region, wherein said peptide comprises at least 7 amino acids;

determining the ability of said APC population to activate said T cell population; and

identifying the T cell epitope in accordance with said determination.

In an embodiment, the peptide further comprises one or more amino acids that are adjacent to (e.g., C and/or N-terminal) the above-mentioned region in the native polypeptide, to permit the detection of epitope spanning adjacent regions. In an embodiment, the peptide comprises from about 1 to about 20 consecutive or contiguous amino acids that are adjacent to (e.g., C and/or N-terminal) the above-mentioned region in the native polypeptide

In an embodiment, the above-mentioned peptide comprises from about 7 to about 25 amino acids, in further embodiments from about 8 to about 25, from about 8 to about 20, from about 8 to about 15 (e.g., 8, 9, 10, 11, 12, 13, 14 or 15) amino acids.

In an embodiment, the above-mentioned amino acids are consecutive or contiguous amino acids. In an embodiment, the above-mentioned peptide comprises a sequence of at least 2, 3, 4, 5, 6, 7, 8, 9, 10, 11, 12, 13, 14, 15, 16, 17, 18, 19, 20, 21, 22, 23, 24 or 25 consecutive/contiguous amino acids from said region.

In an embodiment, a plurality of different peptides comprising a sequence of amino acids from said region are loaded on a plurality of APC populations are used, wherein each of said APC populations is loaded/pulsed with a different peptide. In an embodiment, the above-mentioned plurality of peptides are overlapping peptides encompassing the entire region. The use of overlapping peptides typically permits to more precisely identify/map the epitope. Two adjacent or consecutive peptides may overlap by at least 1, 2, 3, 4, 5, 6, 7, 8, 9, 10, 11, 12, 13, 14, 15, 16, 17, 18, 19, 20, 21, 22, 23, 24 or 25 amino acids. Peptides overlapping by 8 or 13 amino acids are depicted in Table 2 below.

In another aspect, the present invention provides a peptide or peptide identified by the above-mentioned method. In an embodiment, the above-mentioned peptide is a peptide of 50 amino acids or less comprising at least 8 contiguous amino acids from the amino acid sequence of SEQ ID NOs: 3 (AFDERRNKYL), 11 (NGNGDPNNMDKAVKL) or 63 (YRKLKREITF). In an embodiment, the above-mentioned peptide is a peptide of 40, 35, 30, 25, 20, or 15 amino acids or less. In another embodiment, the peptide comprises at least 9, 10, 11, 12, 13, 14 or 15 contiguous amino acids from the amino acid sequence of SEQ ID NOs: 3, 11 or 63. In an embodiment, the above-mentioned peptide comprises, or consists of, the amino acid sequence of SEQ ID NOs: 3, 11 or 63. In a further embodiment, the above-mentioned peptide is a CD4⁺ and/or CD8⁺ T cell epitope, i.e. is capable of activating/stimulating CD4⁺ and/or CD8⁺ T cells under suitable conditions (e.g., in the presence of APCs). In another aspect, the invention provides a vaccine comprising the above-mentioned peptide. The vaccine may further comprise one or more pharmaceutically acceptable adjuvants (which potentiate the immune responses to an antigen and/or modulate it towards the desired immune response) and/or excipients, which are well known in the art. Examples of adjuvants include mineral salts, e.g., aluminium hydroxide and aluminium or calcium phosphate gels; Oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), Montanide ISA-51 and ISA-720 (stabilised water-in-oil emulsion); particulate adjuvants, e.g., virosomes (unilamellar liposomal vehicles incorporating influenza haemagglutinin), ASO4 ([SBAS4] Al salt with MPL), ISCOMS (structured complex of saponins and lipids), polylactide co-glycolide (PLG); microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects); endogenous human immunomodulators, e.g., hGM-CSF or hIL-12 (cytokines that can be administered either as protein or plasmid encoded), Immudaptin (C3d tandem array); as well as inert vehicles, such as gold particles.

### MODE(S) FOR CARRYING OUT THE INVENTION

The present invention is illustrated in further details by the following non-limiting examples.

### EXAMPLE 1

### Materials and Methods

Cells and culture. Peripheral blood mononuclear cells were obtained from a healthy individual, as previously described⁷. B lymphocytes immortalized by Epstein-Barr virus (EBV-B) and CD40-activated B (CD40-B) cells were generated as previously described.⁸

EBV-B or CD40-B cells were sedimented for 15 minutes at 100×g, resuspended in resuspension buffer with 3 μg/10⁶ cells of DNA or mRNA. Cells were electroporated with 1 pulse at 1700V for 20 ms using an MP-100 microporator (Digital-bio, Seoul, Republic of Korea) and resuspended in RPMI 1640 (EBV-B cells) or Iscove's modified Dulbecco's complete (CD40-B cells) medium containing 10% of fetal bovine serum and 2 mM L-glutamine (all from Wisent, St-Bruno, Canada), without antibiotics.

Antigen-specific bulk T cells from peripheral blood mononuclear cells stimulated with autologous CD4O-B cells electroporated with M1 or NP DNA plasmids were cloned by limiting dilution and cultured as previously described.⁸

The gp100-specific CD8⁺ (g209) T-cell clone (described in Dudley M E, et al. J Immunother. 2001 July-August; 24(4):363-73) is specific to native (ITDQVPFSV, SEQ ID NO: 22) and optimized (IMDQVPFSV, SEQ ID NO: 21) versions of the gp100 HLA-A*0201-restricted epitope (209-218). The optimized epitope was used throughout the studies described herein and referred to as gp100^{209-218/2M} or g209.

EBV-B cell lines were cryopreserved in 90% RPMI 1640 complete medium/10% DMSO (Sigma, St-Louis, Mo.), and stored in liquid nitrogen. Antigen-specific T cell clones and CD40-B cells were cryopreserved in 90% FBS (Wisent)/10% DMSO (Sigma), and stored in liquid nitrogen.

HLA typing of donor PBMCs. The HLA genotypes and serotypes of PBMCs were determined by sequencing (Laboratoire d'histocompatibilité, INRS-Institut Armand-Fappier, Laval, Quebec, Canada). HLA genotype of PBMCs from the normal donor was HLA-A*02, 33; B*35, 51; Cw*04, 16; DRB1*04, 11; DQB1*03,03.

cDNA and mRNA preparation. NP and M1 matrix proteins from influenza virus A/Puerto Rico/8/1934/H1N1 strain [Uniprot #P03466 (NP) and P03485 (M1)] cDNA sequences were optimized for improved expression with GeneOptimizer™ from Geneart (Regensberg, Germany) and cloned into pcDNA3.1 (+) plasmid (Invitrogen, Carlsbad, Calif.). Plasmids were transformed into Escherichia coli One Shot TOP 10™ competent cells (Invitrogen) and prepared by plasmid Megaprep™ kit (Qiagen, Hilden, Germany). M1, NP or mock [dickkopf homolog 1 (DKK1)] protein cDNA fragments were amplified by standard PCR from pcDNA3.1 (+)-M1, -NP or -mock (DKK1) with high-fidelity Platinum™ Pfx DNA polymerase (Invitrogen). The primer sets (Integrated DNA technologies, Coralville, Iowa) are listed in Table 1. Nucleotide sequences of the M1⁵⁸⁻⁶⁶ epitope and the g209 epitope were added at the 5′end of some of the M1 and NP fragment reverse DNA primers respectively, before a stop codon (Table 1). PCR conditions of M1 and NP PCR amplification were as follows: 15 min at 95° C., followed by 35 cycles of 45 s at 94° C., 45 s at 55° C. and 90 s at 72° C. The GFX™ PCR DNA and gel band purification kit (GE Healthcare, Waukesha, Wis.) was used to purify PCR-amplified cDNAs when needed according to the manufacturer's instructions.

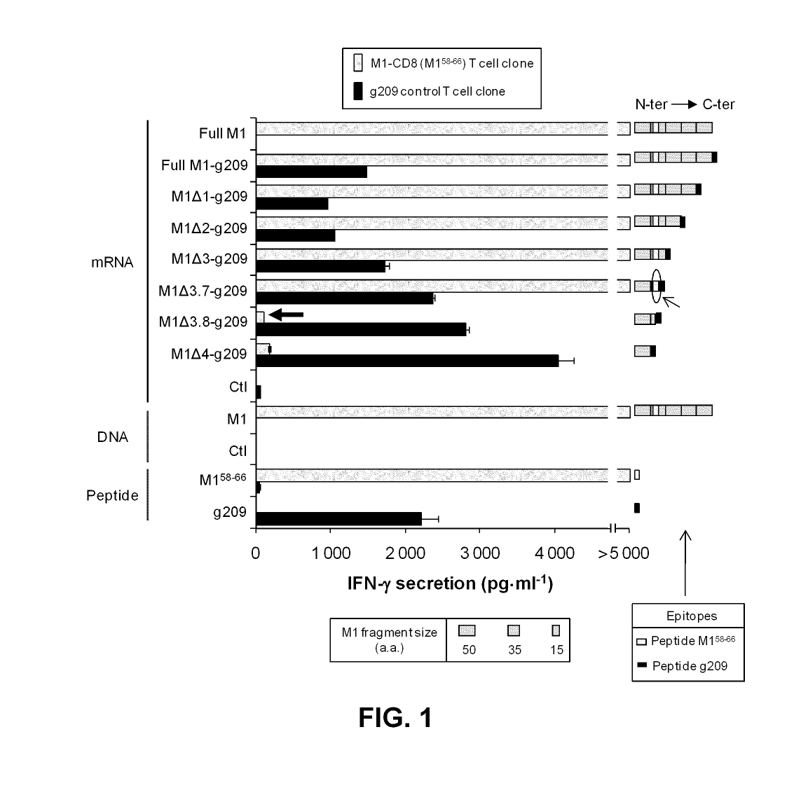

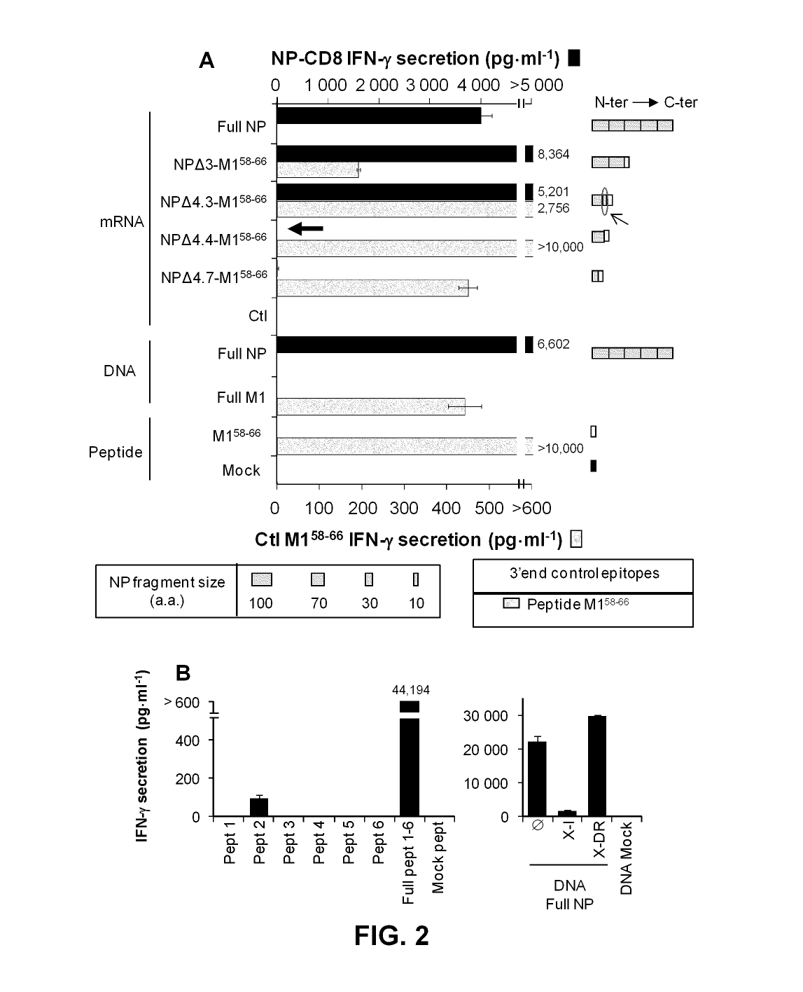

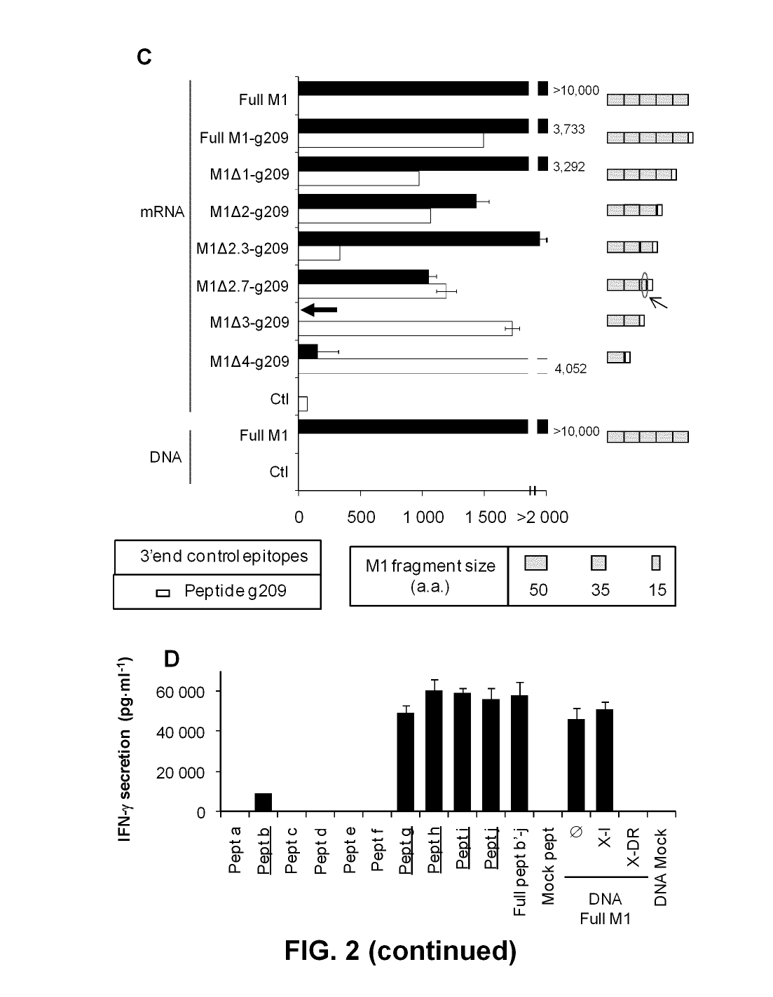

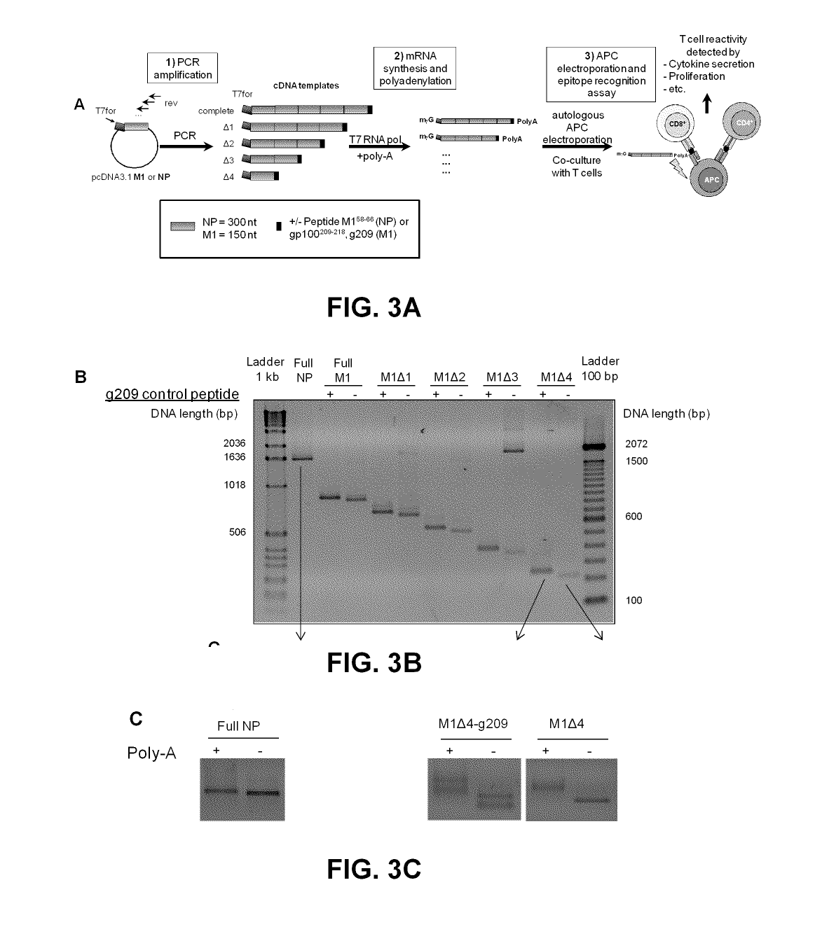

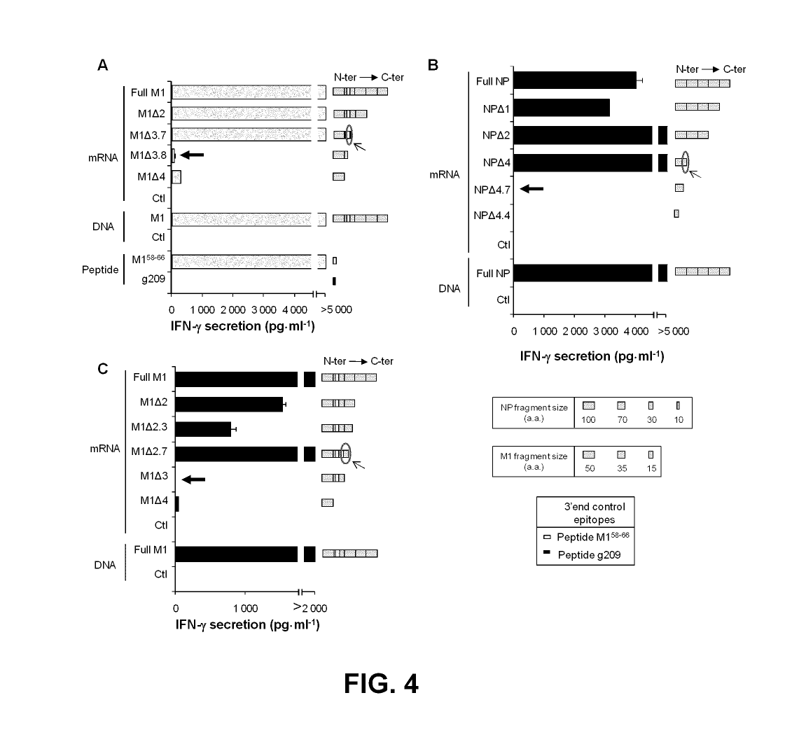

RNA was synthesized in vitro using the mMessage mMachine™, poly(A) tailing and MEGAclear™ kits (Ambion, Austin, Tex.). M1 mRNA fragments were synthesized in vitro from PCR-amplified cDNA amplicons with a high fidelity DNA polymerase as described previously¹⁶. FIG. 3, panel B, shows PCR-amplified M1 cDNA templates on 1.5% agarose gel electrophoresis. M1 cDNA 3′end is shortened by approximately 150 nucleotides between each deletant (approximately 300 nucleotides for NP fragments).The inclusion of a g209 control peptide in the 3′end reverse PCR primer resulted in a minor increase in size of the cDNA templates. When needed, specific cDNA templates (M 1Δ3 and M1Δ1) were isolated on preparative agarose gel and re-amplified by PCR to ensure purity. Finally, RNA synthesis and poly-adenylation were monitored by agarose gel electrophoresis under non-denaturing and non-RNAse-free conditions after migration for 15 min. to minimize RNA degradation in the gel¹⁷ (FIG. 3, panel C). Although fragments of two different sizes were detected for some mRNA fragments, these were most likely due to the remaining secondary structures of RNAs (i.e. M1Δ4-g209 RNA fragment). As RNAs are very sensitive to degradation, it was impossible to confirm beyond doubt that mRNAs were polyadenylated without denaturing conditions and a strict RNAse-free environment. However, integrity of mRNAs was further assessed by control T cell clone recognition (FIGS. 1 and 2A, C).

Synthetic peptides were added to EBV-B cells at a final concentration of 1 to 10 μg/mL for MHC class 110-mer peptides (50 μg/mL for longer peptides) (Table 2) for 3 hours at 37° C. 5% CO₂, and then washed once to remove unbound peptides. T-cell clones were washed and cultured for 4 hours in Iscove's complete medium supplemented with 120 IU/ml of interleukin-2 (IL-2). T cell clones' reactivity to MHC-restricted epitopes was tested on the basis of interferon-γ cytokine secretion as described previously.⁷

### EXAMPLE 2

### Validation of the Method with a Defined Model HLA-A*0201 Epitope from Influenza A Virus Matrix Protein (M1⁵⁸⁻⁶⁶)

PCR-amplified cDNA fragments of various lengths were generated with a T7 promoter forward primer localized at the 5′end of the sequence coding for the defined antigen and a matching 3′end reverse primers ending at different sites in the antigen-coding sequence (FIG. 3, Table 1). From these cDNA fragments, RNA were synthesized and subsequently poly-adenylated (FIG. 3). The resulting mRNA fragments were electroporated into autologous EBV-B, thereby allowing exact allele product matching. Alternatively, autologous CD40-B lymphocytes may also be used as APCs.

TABLE 1
PCR primer sequences for DNA template synthesis. The reverse nucleotide sequence
of the stop codon added at the 3′end of all DNA fragments is in italics. The reverse
nucleotide sequence of M1⁵⁸⁻⁶⁶ peptide added at the 3′end of NP DNA fragments is
underlined. The reverse nucleotide sequence of the g209-2M peptide added at the 3′end
of all M1 DNA fragments is in bold.

| Primer name | Sequence (5′ - 3′) | SEQ ID NO: |
|---|---|---|
| T7 promoter forward | TTAATACGACTCACTATAGGG | 23 |
| (T7for) | | |
| BGH rev | TAGAAGGCACAGTCGAGG | 24 |
| NP revseg M1⁵⁸⁻⁶⁶ | TTACAGGGTGAACACGAAGCCCAGGATGCCGAAGTAG | 25 |
| | CTGCCCTCGT | |
| Nprevseg4 | TTACTGTCCAGCGCTAGCCC | 26 |
| Nprevseg4-M1⁵⁸⁻⁶⁶ | TTACAGGGTGAACACGAAGCCCAGGATGCCCTGTCCA | 27 |
| | GCGCTAGCCC | |
| Nprevseg3A | TTACCGGAAGGGGTCGATGCC | 28 |
| Nprevseg2 | TTATCTCCAAAAATTCCGGT | 29 |
| Nprevseg2-M1⁵⁸⁻⁶⁶ | TTACAGGGTTGAACACGAAGCCCAGGATGCCTCTCCAA | 30 |
| | AAATTCCGGT | |
| Nprevseg1A | TTACAGCTCCCGCATCCACT | 31 |
| Nprevseg0_7-M1⁵⁸⁻⁶⁶ | TTACAGGGTGAACACGAAGCCCAGGATGCCTCCGGC | 32 |
| | GCTGGGGTGTT | |
| Nprevseg0_67 | TTACCGTCTTTCGTCGAAGG | 33 |
| Nprevseg0_67-M1⁵⁸⁻⁶⁶ | TTACAGGGTGAACACGAAGCCCAGGATGCCCCGTCTT | 34 |
| | TCGTCGAAGG | |
| Nprevseg0_33 | TTAGATGTAGAACCGGCCGA | 35 |
| Nprevseg0_33-M1⁵⁸⁻⁶⁶ | TTACAGGGTGAACACGAAGCCCAGGATGCCGATGTAG | 36 |
| | AACCGGCCGA | |
| M1revseg | TTACTTGAACCGCTGCATCT | 37 |
| M1revseg-g209 | TTACACGCTGAAGGGCACCTGGTCCATGATCTTGAAC | 38 |
| | CGCTGCATCT | |
| M1-4revseg | TTAGCTGCTGCCGGCCATCT | 39 |
| M1-4revseg-g209 | TTACACGCTGAAGGGCACCTGGTCCATGATGCTGCT | 40 |
| | GCCGGCCATCT | |
| M1-3revseg | TTAACACACCAGGCCGAAGG | 41 |
| M1-3revseg-g209 | TTACACGCTGAAGGGCACCTGGTCCATGATACACACC | 42 |
| | AGGCCGAAGG | |
| M1-2revseg | TTAGGCCTTGTCCATGTTGT | 43 |
| M1-2revseg-g209 | TTACACGCTGAAGGGCACCTGGTCCATGATGGCCTT | 44 |
| | GTCCATGTTGT | |
| M1-2_7revseg | TTAGTAGATCAGGCCCATGC | 45 |
| M1-2_7revseg-g209 | TTACACGCTGAAGGGCACCTGGTCCATGATGTAGATC | 46 |
| | AGGCCCATGC | |
| M1-2_3revseg | TTACTCTTTGGCGCCGTGGA | 47 |
| M1-2_3revseg-g209 | TTACACGCTGAAGGGCACCTGGTCCATGATCTCTTTG | 48 |
| | GCGCCGTGGA | |
| M1-1revseg | TTACAGCCATTCCATCAGCA | 49 |
| M1-1revseg-g209 | TTACACGCTGAAGGGCACCTGGTCCATGATCAGCCAT | 50 |
| | TCCATCAGCA | |
| M1-1_3revseg | TTACAGGGTGAACACGAAGC | 51 |
| M1-1_3revseg-G209 | TTACACGCTGAAGGGCACCTGGTCCATGATCAGGGT | 52 |
| | GAACACGAAGC | |
| M1-1_2revseg | TTACTTGGTCAGGGGGCTCA | 53 |
| M1-1_2revseg-g209 | TTACACGCTGAAGGGCACCTGGTCCATGATCTTGGTC | 54 |
| | AGGGGGCTCA | |

The mPEC method was first validated with CD8⁺ T lymphocytes (M1-CD8) specific to a defined model HLA-A*0201 epitope from influenza A virus matrix protein (M1⁵⁸⁻⁶⁶). mRNA encoding the full length M1 protein was recognized by M1⁵⁸⁻⁶⁶-specific T cells, and successive deletions at the C-terminal end were recognized until the epitope was specifically deleted (FIG. 1, light grey, upper bars), corresponding to M1Δ3.8 fragment. Conversely, the M1Δ3.7 fragment, which ends immediately after the epitope sequence, was well recognized. This shows the accuracy of the mPEC method.

Particularly, mRNA of poor quality or degraded mRNA can result in no or low protein production by the APCs, which could in turn fail to elicit a T-cell response, thus providing a false-negative signal. To control for mRNA integrity and protein translation after electroporation, a sequence coding for a known peptide which can be recognized by available T lymphocytes was added at the 3′end of mRNAs sequence. For M1 fragments, the glycoprotein (gp)100 HLA-A*0201 epitope (gp100²⁰⁹⁻²¹⁸) was added. Gp100-specific T lymphocytes specifically recognized all M1 constructs bearing the gp100²⁰⁹⁻²¹⁸ epitope (FIG. 1, black, lower bars), confirming the integrity of the M1 mRNA fragments. As negative specificity controls, gp100²⁰⁹⁻²¹⁸-specific T cells did not recognize full M1 mRNA (without the gp100²⁰⁹⁻²¹⁸ epitope), and M1⁵⁸⁻⁶⁶-specific T cells did not recognize EBV-B cells pulsed with the gp100²⁰⁹⁻²¹⁸ peptide.

### EXAMPLE 3

### Identification of Novel MHC Class I and II Epitopes Using the mPEC Method

Two previously unknown MHC classes I and II epitopes derived from model influenza targets with CD8⁺ T lymphocytes specific to influenza A nucleoprotein (NP-CD8), and CD4⁺ T lymphocytes specific to M1 (M1-CD4), were identified by the mPEC method. As shown by interferon-γ secretion, the NP-CD8 T cell clone failed to respond to the mRNA fragment NPΔ4.4 whereas NPΔ4.3 was well recognized. Similar results were obtained by measuring MIP-1β secretion. The control M1⁵⁸⁻⁶⁶ peptide added at the 3′end of NP mRNAs (FIG. 2A) was recognized by relevant T cells, showing mRNA fragment integrity. This showed that the NP-CD8 epitope was localized in the deletion between fragments NPΔ4.3 and NPΔ4.4, corresponding to an 11 amino acid sequence (positions 68 to 78, FIG. 2A). To these 11 residues, 8 amino acids from NPD4.4 fragment were added at the N-terminal end to account for a loss of a potential epitope spanning both NPΔ4.3 and NPΔ4.4, and 6 overtlaping peptides of 10-mer each covering this sequence were synthesized (Table 2). The whole 19-mer peptide was well recognized by the NP-CD8 T-cell clone. Among the 10-mer peptides, only peptide 2 (AFDERRNKYL, SEQ ID NO:3) was more weakly but nevertheless specifically recognized (FIG. 2B, indicating that it contains the NP-CD8-specific epitope (or at least a major part thereof) but additional amino acid trimming and sequence optimization would permit to identify the exact epitope recognized.

TABLE 2
Peptides synthesized to test NP-CD8 and M1-CD4 T cell clone specificity with the
mPEC method. Recognized T cell-specific epitopes are underlined. Amino acids were
added at the N-terminal of peptides to account for a potential epitope spanning
both NPΔ4.3 and NPΔ4.4 mRNA fragments (in italics).

| | | SEQ ID NO: |
|---|---|---|
| NP-CD8 peptides | | |
| Peptide 1-6 | LSAFDERRNKYLEEHPSAG | 1 |
| Peptide 1 | LSAFDERRNK | 2 |
| Peptide 2 | AFDERRNKYL | 3 |
| Peptide 3 | DERRNKYLEE | 4 |
| Peptide 4 | RRNKYLEEHP | 5 |
| Peptide 5 | NKYLEEHPSA | 6 |
| Peptide 6 | KYLEEHPSAG | 7 |
| M1-CD4 peptides | | |
| Peptide a-j | ALNGNGDPNNMDKAVKLYRKLKREITFHGAKE | 8 |
| Peptide b-j | GNGDPNNMDKAVKLYRKLKREITFHGAKE | 9 |
| Peptide a | ALNGNGDPNNMDKAV | 10 |
| Peptide b | NGNGDPNNMDKAVKL | 11 |
| Peptide c | NGDPNNMDKAVKLYR | 12 |
| Peptide d | DPNNMDKAVKLYRKL | 13 |
| Peptide e | NNMDKAVKLYRKLKR | 14 |
| Peptide f | MDKAVKLYRKLKREI | 15 |
| Peptide g | KAVKLYRKLKREITF | 16 |
| Peptide h | VKLYRKLKREITFHG | 17 |
| Peptide i | LYRKLKREITFHGAK | 18 |
| Peptide j | YRKLKREITFHGAKE | 19 |

| | |
|---|---|
| M1⁵⁸⁻⁶⁶ peptide | |
| GILGFVFTL | 20 |
| G209-2M peptide | |
| IMDQVPFSV | 21 |

The mPEC method is also effective for the identification of MHC class II epitopes (or CD4⁺ T cell epitope). The MHC class II M1-CD4 T cell epitope is localized between the M1Δ2.7 and M1Δ3 constructs. A series of overlapping peptides were constructed based on the 18 amino acid sequence specifically deleted between these 2 fragments, to which 8 amino acids from M1Δ3 fragment at the N-terminal end were added to account for potential loss of the P9 amino acid of the core MHC class II epitope. 5 amino acids from M1Δ3 fragment were further added at the N-terminal end to account for the loss of a potentially important flanking region of the MHC class II epitope¹⁸. 10 overlapping 15-mer peptides encompassing this sequence (Table 2) were synthesized, from which 2 HLA-DR-restricted MHC class II epitopes were recognized by the M1-CD4⁺ T cell clone (FIG. 2D and Table 2). More particularly, a 10-mer HLA-DR-restricted MHC class II epitope (YRKLKREITF, SEQ ID NO:63) localized between the M1Δ2.7 and M1Δ3 constructs was specifically recognized by the M1-CD4 T-cell clone. M1-CD4 T cells also weakly recognized the 15-mer Peptide b, which could represent an alternative epitope or heterogeneity in the T-cell clone. Hence, mPEC allows for the identification of MHC class II epitopes.

### EXAMPLE 4

### Use of CD40-Activated B Lymphocytes (CD40-B) as APCs in the mPEC Method

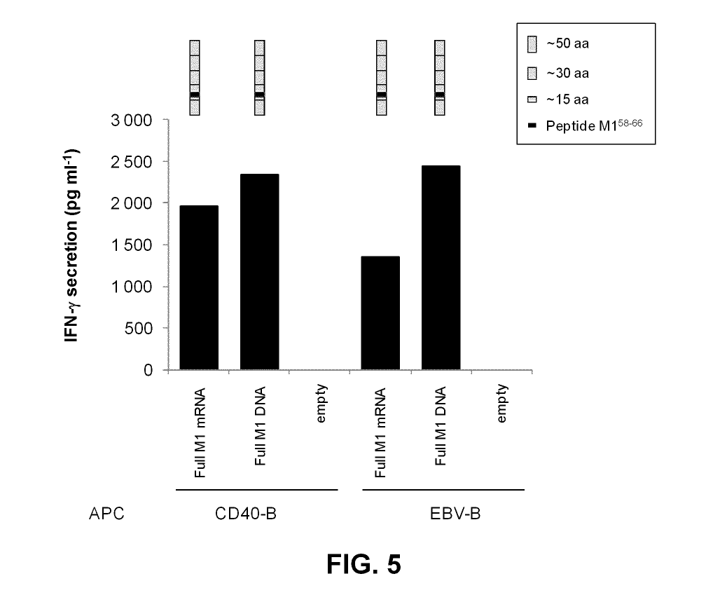

CD40-activated B lymphocytes (CD40-B) can serve as alternative autologous APCs. CD40-B and EBV-B cells were electroporated with M1-coding DNA plasmids or mRNA prepared from PCR-amplified M1 cDNA and co-cultured with M1⁵⁸⁻⁶⁶ (M1-CD8) T cells. Both EBV-B and CD40-B cells resulted in comparable IFN-γ production by M1⁵⁸⁻⁶⁶ T cells (FIG. 5). Considering that CD40-B can be generated more rapidly as compared to EBV-B cells (10-15 days compared to 3-6 weeks), these cells represent an interesting alternative to EBV-B cells.

Although the present invention has been described hereinabove by way of specific embodiments thereof, it can be modified, without departing from the spirit and nature of the subject invention as defined in the appended claims.

### REFERENCES

1. Soethout E C, Meiring H D, Jong A Pd, et al. Identifying the epitope-specific T cell response to virus infections. Vaccine. 2007; 25:3200-3203.
2. Pilla L, Rivoltini L, Patuzzo R, et al. Multipeptide vaccination in cancer patients. Expert Opin Biol Ther. 2009; 9:1043-1055.
3. Bakker A, Hoppes R, Linnemann C, et al. Conditional MHC class I ligands and peptide exchange technology for the human MHC gene products HLA-A1, -A3, -A11, and -B7. Proc Natl Acad Sci USA. 2008; 105:3825-3830.
4. Engelhard V H. The contributions of mass spectrometry to understanding of immune recognition by T lymphocytes. Int J Mass Spectrom. 2007; 259:32-39.
5. Robbins P, el-Gamil M, Li Y, et al. Cloning of a new gene encoding an antigen recognized by melanoma-specific HLA-A24-restricted tumor-infiltrating lymphocytes. J Immunol. 1995; 154:5944-5950.
6. Britten C M, Meyer R G, Graf C, et al. Identification of T cell epitopes by the use of rapidly generated mRNA fragments. J Immunol Methods. 2005; 299:165-175.
7. Pelletier S, Tanguay S, Lee S, et al. TGF-alpha as a candidate tumor antigen for renal cell carcinomas. Cancer Immunol Immunother. 2009; 58:1207-1218.
8. Lapointe R, Bellemare-Pelletier A, Housseau F, et al. CD40-stimulated B lymphocytes pulsed with tumor antigens are effective antigen-presenting cells that can generate specific T cells. Cancer Res. 2003; 63:2836-2843.
9. Zhang Q, Wang P, Kim Y, et al. Immune epitope database analysis resource (IEDB-AR). Nucleic Acids Res. 2008; 36: W513-W518.
10. Ho O, Green W R. Alternative translational products and cryptic T cell epitopes: expecting the unexpected. J Immunol. 2006; 177:8283-8289.
11. Skipper J, Hendrickson R, Gulden P, et al. An HLA-A2-restricted tyrosinase antigen on melanoma cells results from posttranslational modification and suggests a novel pathway for processing of membrane proteins. J Exp Med. 1996; 183:527-534.
12. Hanada K-I, Yewdell J W, Yang J C. Immune recognition of a human renal cancer antigen through post-translational protein splicing. Nature. 2004; 427:252-256.
13. Fairchild P J, Pope H, Wraith D C. The nature of cryptic epitopes within the self-antigen myelin basic protein. Int Immunol. 1996; 8:1035-1043.
14. Yewdell J W, Bennink J R. Immunodominance in major histocompatibility complex class I-restricted T lymphocyte responses. Annu Rev Immunol. 1999; 17:51-88.
15. Contreras C E, Ploton I N, Siliciano R F, et al. Mapping of specific and promiscuous HLA-DR-restricted T-cell epitopes on the Plasmodium falciparum 27-kilodalton sexual stagespecific antigen. Infect Immun. 1998; 66:3579-3590.
16. Nishioka M, Mizuguchi H, Fujiwara S, et al. Long and accurate PCR with a mixture of KOD DNA polymerase and its exonuclease deficient mutant enzyme. J Biotechnol. 2001; 88:141-149.
17. Euler H V, Khoshnoud R, He Q, et al. Time-dependent RNA degradation affecting cDNA array quality in spontaneous canine tumours sampled using standard surgical procedures. Int J Mol Med. 2005; 16:979-985.
18. Godkin A J, Smith K J, Willis A, et al. Naturally processed HLA Class II peptides reveal highly conserved immunogenic flanking region sequence preferences that reflect antigen processing rather than peptide-MHC interactions. J Immunol. 2001; 166:6720-6727.
19. Lepage S and Lapointe R. Melanosomal targeting sequences from gp100 are essential for MHC class II-restricted endogenous epitope presentation and mobilization to endosomal compartments. Cancer Res. 2006; 66:2423-32.
20. Wang R-F. Identification of MHC class II-restricted tumor antigens recognized by CD4⁺ T cells. Methods. 2003; 29:227-235.
21. U.S. Patent Publication No. 2008/0311098.

1. A method for determining whether a region of a polypeptide of interest comprises one or more T cell epitopes, said method comprising:
(a) providing an mRNA comprising a first domain encoding said region, wherein said mRNA is obtained by in vitro transcription of a DNA encoding said region, and wherein said DNA is obtained by nucleic acid amplification using one or more oligonucleotides hybridizing to a nucleic acid encoding said polypeptide of interest or to the complement thereof;
(b) introducing said mRNA into an antigen-presenting cell (APC) population;
and (c) determining the ability of said APC population to activate a first T cell population;
wherein activation of said first T cell population by said APC population is indicative that said region comprises one or more T cell epitopes.

2-3. (canceled)

4. The method of claim 1, wherein said region comprises from about 10 to about 100 amino acids.

5. (canceled)

6. The method of claim 1, wherein said mRNA further comprise a second domain encoding a detectable moiety, and wherein said method further comprises determining the presence of said detectable moiety.

7. The method of claim 6, wherein said detectable moiety is a known T cell epitope, and wherein said method further comprises determining the ability of said APC populations to activate a second T cell population recognizing said known T cell epitope.

8. The method of claim 7, wherein said second domain is located 3′ relative to said first domain.

9. The method of claim 1, wherein said mRNA further comprising a poly(A) tail.

10. The method of claim 1, wherein said APC is a B-cell.

11. The method of claim 1, wherein said first T cell population is a T cell clone.

12. (canceled)

13. The method of claim 1, wherein said APC population and said first T cell population are autologous.

14. (canceled)

15. A method for determining whether a region of a polypeptide of interest comprises one or more T cell epitopes, said method comprising:
(a) providing a first mRNA comprising a first domain encoding said polypeptide of interest or a fragment thereof comprising said region;
(b) providing a second mRNA comprising the first domain of said first mRNA but in which the portion encoding said region is lacking, wherein said first and second mRNA are obtained by in vitro transcription of DNAs encoding said polypeptide of interest or fragment thereof, and wherein said DNAs are obtained by nucleic acid amplification using oligonucleotides hybridizing to different portions of a nucleic acid encoding said polypeptide of interest or a complement thereof;
(c) introducing said first and second mRNAs into first and second antigen-presenting cell (APC) populations, respectively; and
(d) determining the ability of said first and second APC populations to activate a first T cell population;
wherein a higher activation of said first T cell population by said first APC population relative to said second APC population is indicative that said region comprises one or more T cell epitopes.

16-17. (canceled)

18. The method of claim 15, wherein said region comprises from about 10 to about 100 amino acids.

19. (canceled)

20. The method of claim 15, wherein said second mRNA encodes a C-terminal deletion mutant of the polypeptide of interest or fragment thereof of (a).

21. The method of claim 15, wherein said first and second mRNAs further comprise a second domain encoding a known T cell epitope, and wherein said method further comprises determining the ability of first and second APC populations to activate a second T cell population recognizing said known T cell epitope.

22. The method of claim 15, wherein said mRNA further comprising a poly(A) tail.

23. The method of claim 15, wherein said APC is a B-cell.

24. The method of claim 15, wherein said first T cell population is a T cell clone.

25. (canceled)

26. The method of claim 15, wherein said APC populations and said first T cell population are autologous.

27. (canceled)

28. A method for identifying one or more T cell epitopes in a polypeptide of interest, said method comprising:
(a) performing the method of claim 1 to identify a region of said polypeptide comprising said one or more T cell epitopes;
(b) contacting a T cell population with an antigen-presenting cell (APC) population loaded or pulsed with a peptide comprising a sequence of amino acids from said region, wherein said peptide comprises at least 7 amino acids;
(c) determining the ability of said APC population to activate said T cell population; and
(d) identifying the T cell epitope in accordance with said determination.

29. The method of claim 28, wherein a plurality of different peptides comprising amino acids located within said region loaded on a plurality of APC populations are used, wherein each of said APC populations is loaded with a different peptide.

30. (canceled)

31. A peptide of 50 amino acids or less comprising at least 8 contiguous amino acids from the amino acid sequence of SEQ ID NOs: 3, 11 or 63.
